(19) United States
(12) Patent Application Publication
(10) Pub. No.: US 2017/0095214 A1
(43) Pub. Date: Apr. 6, 2017

(54) APPARATUSES AND METHODS FOR DETERMINING WHETHER CARDIOPULMONARY RESUSCITATION IS CONDUCTED BASED ON AN IMPEDANCE SIGNAL

(71) Applicant: UNIVERSITY OF WASHINGTON, Seattle, WA (US)

(72) Inventors: ALAMPALLAM R. RAMACHANDRAN, Seattle, WA (US); Jason Couit, Seattle, WA (US); Lawrence D. Sherman, Seattle, WA (US); Peter J. Kudenchuk, Seattle, WA (US)

(73) Assignee: UNIVERSITY OF WASHINGTON, Seattle, WA (US)

(21) Appl. No.: 15/317,070

(22) PCT Filed: Jun. 26, 2015
(86) PCT No.: PCT/US15/38023

§ 371 Date: Dec. 7, 2016

Related U.S. Application Data
(60) Provisional application No. 62/017,497, filed on Jun. 26, 2014.

(51) Int. Cl. A61B 5/00; A61M 16/00; A61B 5/04; A61N 1/08; A61B 5/0402; A61N 1/04

(52) U.S. Cl. CPC A61B 5/7257; A61B 5/0402; A61B 5/6823; A61N 1/046; A61B 5/04012; A61B 5/725; A61B 5/746; A61N 1/08; A61M 16/0048

(57) ABSTRACT

Examples of systems, apparatuses, and methods for determining whether CPR is being conducted based on an impedance signal are described. An example system may include a defibrillator including a cardiopulmonary resuscitation (CPR) analyzer configured to detect an impedance signal between electrodes applied to a chest of a patient. The CPR analyzer may be further configured to transform the impedance signal to a frequency domain representation to provide transformed frequency data, and to detect peaks within the transformed frequency data. The CPR analyzer may be further configured to classify the impedance signal as one of CPR or no CPR based on the detected peaks. The CPR analyzer may be further configured to determine a chest compression rate based on the detected peaks.

### CROSS-REFERENCE TO RELATED APPLICATION(S)

This application claims the benefit of the earlier filing dates of U.S. Provisional Application No. 62/017,497, filed Jun. 26, 2014, entitled “Detection of Chest Compressions Using the Amplitude and Frequency Characteristics of the Chest's Electrical Impedance,” which is hereby incorporated by reference in its entirety for any purpose.

### BACKGROUND

Cardiopulmonary resuscitation (CPR), combined with defibrillation, is an essential treatment of cardiac arrest and involves chest compressions designed to perfuse the heart, brain and other organs during the arrest. In some instances, automatic external defibrillators (AED) may be designed to analyze an electrocardiogram (ECG) signal during a cardiac arrest through two electrode pads attached to the chest of the patient in order to determine whether to provide a shock to the patient via the two pads. An ECG signal provides an indication of electrical activity of the heart. The two pads attached to the patient may detect electrical pulses generated by the polarization and depolarization of cardiac tissue, and translates the electrical pulses into a waveform. The waveform can be used to measure rate and regularity of heartbeats, the presence of any damage to the heart, and the effects of drugs or devices used to regulate the heart.

During a cardiac arrest, an AED may analyze the ECG signal to detect whether the patient's heart is exhibiting a shockable ECG rhythm. An example of a shockable rhythm may include ventricular fibrillation (i.e., a condition where there is uncoordinated contraction of the cardiac muscle of the ventricles of the heart, causing the cardiac muscles to quiver rather than contract in a coordinated fashion). Examples of non-shockable ECG rhythms may include asystole (i.e., flatline or state of no cardiac electrical activity), organized cardiac electrical activity (including rhythms that produce blood flow), or pulseless electrical activity (i.e., electrical signals indicate heart rhythm, but no pulse is produced). Thus, prior to delivering a shock, an AED must first determine if the underlying ECG signal indicates a shockable rhythm with reasonable certainty, to avoid administering a shock to a patient with a non-shockable rhythm.

Conventional AEDs instruct a responder to provide CPR chest compressions and artificial ventilation during the arrest. Provision of CPR introduces artifacts into an ECG signal, obscuring the ability of the AED to detect an ECG rhythm of the heart of the patient. Some conventional AEDs may periodically require the responder to cease CPR (e.g., for 7 or more seconds) to allow for analysis of the ECG rhythm via the ECG signal. Cessation of CPR for analysis, even for a short while, may significantly reduce chances of survival due to, among other issues, loss of perfusion pressure.

Other conventional AEDs may use a separate “puck” device with force and acceleration sensors that is placed between the patient and the hands of the person administering CPR to detect CPR compressions, and analyze the ECG rhythm via the ECG signal using different algorithms based on whether CPR is detected or not. The “puck” being a separate device, may increase cost and complexity, and may lend itself to not being consistently used.

### SUMMARY

Examples of systems, apparatuses, and methods for determining whether cardiopulmonary resuscitation is being conducted based on an impedance signal are described herein. An example system may include a defibrillator including a cardiopulmonary resuscitation (CPR) analyzer configured to detect an impedance signal between electrodes applied to a chest of a patient. The CPR analyzer may be further configured to transform the impedance signal to a frequency domain representation to provide transformed frequency data, and to detect peaks within the transformed frequency data. The CPR analyzer may be further configured to classify the impedance signal as one of CPR or no CPR based on the detected peaks.

An example method may include transforming an impedance signal that indicates impedance between electrodes applied to a chest of a patient to a frequency domain representation to provide transformed frequency data. The example method may further include detecting peaks within the transformed frequency data. The peaks have a bandwidth that is less than a bandwidth threshold. The example method may further include identifying a highest peak and secondary peaks, and classify the impedance signal as CPR or no CPR based whether the highest peak or the secondary peaks are located within a defined frequency range.

Another example method may include transforming a clip of an impedance signal that indicates impedance between electrodes applied to a chest of a patient to a frequency domain representation to provide transformed frequency data. The example method may further include identifying, within the transformed frequency data, a highest peak having a bandwidth that meets a bandwidth threshold. The example method may further include, responsive to the highest peak being located within a defined frequency range, classifying the clip as cardiopulmonary resuscitation (CPR). The example method may further include responsive to the highest peak being located outside the defined frequency range, classifying the clip as CPR or no CPR based on secondary peak located within the defined frequency range.

### BRIEF DESCRIPTION OF THE DRAWINGS

The foregoing and other features of the present disclosure will become more fully apparent from the following description and appended claims, taken in conjunction with the accompanying drawings. Understanding that these drawings depict only several examples in accordance with the disclosure and are, therefore, not to be considered limiting of its scope, the disclosure will be described with additional specificity and detail through use of the accompanying drawings, in which:

FIG. 1 is an exemplary illustration of an automatic external defibrillator system applied to a patient according to an embodiment of the present disclosure.

FIG. 2 is a block diagram of a defibrillation system according to an embodiment of the present disclosure.

FIG. 3 is a block diagram of a CPR analyzer according to an embodiment of the present disclosure.

FIG. 4 is a flow chart of an exemplary method for classifying an impedance signal as CPR detected or no CPR detected according to an embodiment of the present disclosure.

FIG. 5 is a flow chart of an exemplary method for classifying an impedance signal based on identified peaks as CPR detected or no CPR detected according to an embodiment of the present disclosure.

FIG. 6 is a block diagram illustrating example peak configurations in the frequency domain representation according to an embodiment of the present disclosure.

FIG. 7 is a block diagram illustrating an example computing device that includes a CPR analyzer according to an embodiment of the present disclosure.

### DETAILED DESCRIPTION

In the following detailed description, reference is made to the accompanying drawings, which form a part hereof. In the drawings, similar symbols typically identify similar components, unless context dictates otherwise. The illustrative examples described in the detailed description, drawings, and claims are not meant to be limiting. Other examples may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are implicitly contemplated herein.

Examples described herein relate generally to apparatuses, systems, and methods for determining whether chest compressions during CPR are being administered by analyzing an impedance signal between two electrodes on the chest of a patient. While the examples described herein are primarily discussed in the context of automatic external defibrillators, it will be understood that the apparatuses, systems, and methods disclosed are equally applicable and can be used in the context of any other therapeutic or clinical device, such as with hospital monitors, implantable defibrillators, or other defibrillators with a capability of determining whether CPR is being administered by analyzing an impedance signal between two electrodes attached to a chest of a patient. Generally, examples of the present disclosure may be used with any device capable of monitoring an ECG signal via electrodes attached to the chest of a patient. Examples of the present disclosure may also be used to perform a post hoc analysis of impedance signal data collected from one or more patients. The post hoc analysis may be used in determining quality of medical care, such as CPR quality metrics (e.g., consistency, timeliness, etc.). Accordingly, the particular examples provided herein are for illustration purposes only and are not to be taken in a limiting sense.

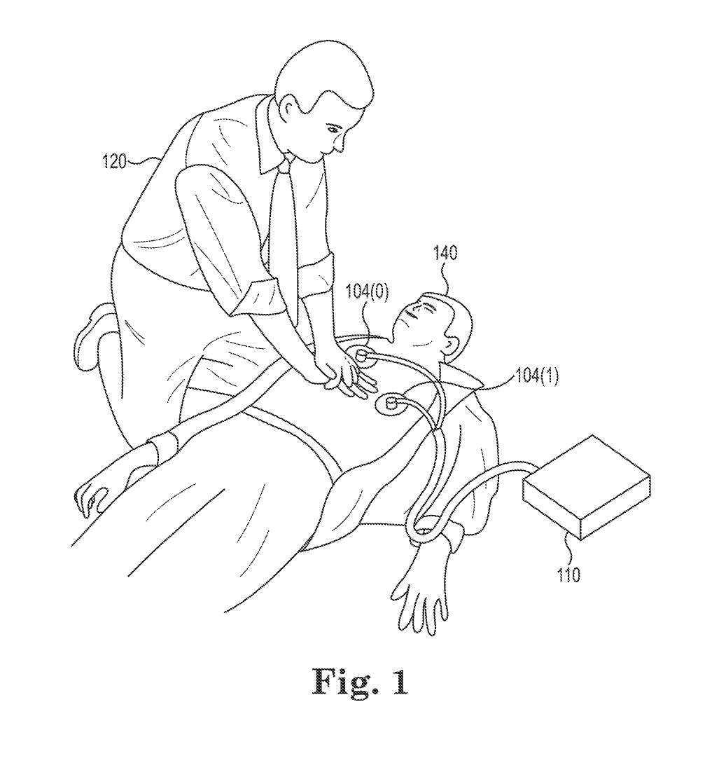

FIG. 1 is an illustration of a responder 120 performing CPR on a patient 140 that is connected to an AED 110. In this scenario, the patient 140 may be exhibiting signs of cardiac arrest. The responder 120 may be a person that is trained in proper CPR techniques. In this example, the patient 140 may have two electrodes 104(0-1) applied to his/her chest. The two electrodes 104(0-1) may be attached to the skin of the patient 140 at conventional locations, such as one electrode 104(0) applied under the right collar bone and the other electrode 104(1) applied to left lower chest. The two electrodes 104(0-1) may be coupled to the AED 110 via a cable.

The AED 110 may detect an ECG signal from the patient 140 via the two electrodes 104(0-1), including while the responder 120 is performing CPR. The AED 110 may analyze the ECG signal to classify the ECG rhythm of the patient 140 as shockable or non-shockable. Responsive to the classification of a shockable rhythm, the AED 110 may apply high-voltage (e.g. 1,300-1,800 volts) shocks. While the AED is connected to the two electrodes 104(0-1) to detect the ECG signal, the responder 120 may perform CPR by applying downward forces or compressions to the sternum of the patient 140. In some instances, CPR may also include the responder 120 blowing air into the mouth or nose of the patient 140 by mouth-to-mouth or mouth-to-nose breathing. Analysis of the EGC signal may be dependent on whether CPR is being administered to the patient 140. That is, analysis of the ECG signal may be different when CPR is being administered than when no CPR is being administered. Thus, the AED 110 may further analyze an impedance signal between the electrodes 104(0-1) over time to provide a CPR/no CPR classification associated with the patient 140. In some examples, the AED 110 may prompt the responder 120 to stop CPR to allow for a shock to be administered to the patient 140.

The AED 110 may include an CPR analyzer to determine whether CPR is being administered to the patient 140 based on analysis of an impedance signal between the electrodes 104(0-1) over time. This information may be used to assist in analysis of the ECG signal, or to provide reminders to the responder 120 to administer CPR. In analyzing an ECG signal, chest compressions during CPR may introduce artifacts into the ECG signal, which may mask or obscure the underlying ECG rhythm, making classification of an ECG rhythm of the patient 120 difficult, and thus the AED 110 may apply signal processing techniques to the impedance signal to determine whether CPR is being administered. The analysis may include transforming the impedance signal to a frequency domain representation, and identifying locations of peaks in the frequency domain representation. The location of the peaks may be used to determine whether CPR is being administered.

In some embodiments, the CPR analyzer may include other or different decision-making methodologies. While the above describes a determination of whether CPR is being administered in an AED 110, the determination may be performed in other devices, such as an implantable defibrillator or an ECG monitor in a hospital setting that constantly or periodically monitors ECG signals via electrodes for evaluations over time or during a medical event.

While AED 110 is described as an automatic external defibrillator, which is generally designed for small physical size, light weight, and relatively simple user interface capable of being operated by personnel without high training levels, in other embodiments, the AED 110 may additionally or alternatively include other defibrillators, such as a manual defibrillator, an implantable defibrillator, a paramedic defibrillator, and/or a clinical defibrillator. Generally, paramedic or clinical defibrillators may be carried by an emergency medical service (EMS) responder, and tend to be larger, heavier, and have a more complex user interface capable of supporting a larger number of manual monitoring and analysis functions.

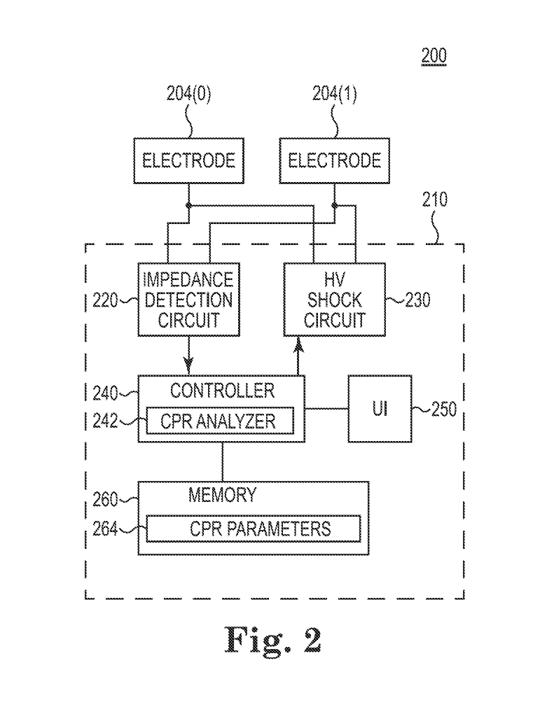

FIG. 2 is a block diagram of defibrillation system 200 according to an embodiment of the disclosure. The defibrillation system 200 may include a pair of electrodes 204(0-1) coupled to an AED 210. The AED 210 may be implemented in the AED 110 of FIG. 1.

The AED 210 may be include an impedance detection circuit 220 coupled to the pair of electrodes 204(0-1). The pair of electrodes 204(0-1) may be connected across the chest of a patient, such as the patient 140 of FIG. 1. The impedance detection circuit 220 may sense impedance between the pair of electrodes 201(0-1). In an example, the impedance may be sensed by providing a signal having a higher frequency than an ECG signal. The signal may be modulated down to a near-zero rate. The impedance detection circuit 220 may provide the impedance signal between the pair of electrodes 204(0-1) to the controller 240.

The controller 240 may include a CPR analyzer 242. The CPR analyzer 242 may transform and filter the impedance signal from the impedance detection circuit 220, and may detect peaks in the signal. The CPR analyzer 242 may determine whether CPR is being administered based on the detected peaks. The CPR analyzer 242 may provide a CPR/no CPR classification of the impedance signal to the controller 240. The controller 240 may analyze the ECG signal using an algorithm that is selected based on the CPR/no CPR classification. The controller 240 may classify the ECG rhythm of the ECG signal of the patient as shockable or non-shockable. If a shockable rhythm is detected (e.g., in combination with determination of a treatment regimen that indicates immediate defibrillation shock), the controller 240 may send a signal to high voltage (HV) shock circuit 230 to charge in preparation for delivering a shock. The AED 210 may include a user interface 250 that provides an indication to the controller 240 to administer the shock responsive to a user input. The user interface 250 may also provide an indication to the responder to begin or resume provision of CPR responsive to a signal from the controller 240 indicating that no CPR is detected by the CPR analyzer 242. The AED 210 may further include a memory 260 that is configured to store impedance parameters 264 used for the classification by the CPR analyzer 242.

In operation, the pair of electrodes 204(0-1) may be attached to a patient experiencing a medical event, such as cardiac arrest. The impedance detection circuit 220 may provide an impedance signal across the pair of electrodes 204(0-1) to continuously monitor impedance between the pair of electrodes 204(0-1), including while the patient is receiving CPR or other medical care. The impedance detection circuit 220 may provide the sensed impedance to the controller 240. The CPR analyzer within the controller 240 may apply signal processing techniques the impedance signal transform and filter the signal. The CPR analyzer 242 may further identify peaks in the transformed and filtered signal, and may determine based on the identified peaks whether CPR is being administered. The CPR analyzer 242 may provide an indication as to whether CPR is being administered to the controller 240. The controller 240 may analyze an ECG signal of the patient received via the pair of electrodes 204(0-1) to classify the ECG rhythm of the patient as shockable or non-shockable. An example of a shockable rhythm may include ventricular fibrillation. Examples of non-shockable rhythms may include asystole (e.g., flatline or state of no cardiac electrical activity), organized cardiac activity (e.g., normal sinus rhythm), or pulseless electrical activity (e.g., electrical signals indicate heart rhythm, but no pulse is produced). The algorithm used in the analysis of the ECG signal may be based on whether CPR is being administered or not. If a shockable classification is determined, the controller 240 may send a command to the HV shock circuit 230 to begin charging. Responsive to an input at the user interface 250, the HV shock circuit 230 may release the high voltage to the electrodes 204(0-1) to administer a shock to a patient.

The CPR analyzer 242 may analyze sliding windows (e.g., clips) of the impedance signal from the impedance detection circuit 220, and may classify each clip as chest compression administration or no chest compression administration. The clips may overlap or may be independent. In an example, the CPR analyzer 242 may execute the classification algorithm at one second intervals, which each clip being 6 seconds long. The chest compression classification may be allocated to a mid-point of the respective clip (e.g., a middle one second of the clip when executed every second). In an embodiment, the CPR analyzer 242 algorithm may filter and transform each clip. For example, the CPR analyzer 242 may apply an amplitude weighting window function (e.g., Tukey, Hamming, Hanning, etc.) to reduce a side lobe effect of the clip. The CPR analyzer 242 may detrend the windowed clip to remove a DC offset and linear trend. The CPR analyzer 242 may apply a bandpass filter to the detrended clip to remove artifacts from the clip outside the pass frequencies, such as an ECG signal or other noise. The CPR analyzer 242 may transform the bandpass filtered clip to a frequency domain representation by applying a discrete Fourier transform (DFT) to provide transformed frequency data.

The CPR analyzer 242 may detect all of the peaks that meet a bandwidth threshold within the transformed frequency data. The bandwidth threshold may be based on a bandwidth between points of the peak. For example, the bandwidth threshold may be a half-amplitude bandwidth, and the identified peak may have a half-amplitude bandwidth less than the bandwidth threshold. In an example, the bandwidth threshold may be 0.6 Hz. A more narrow bandwidth of the identified highest peak may indicate a sinusoid-like trace indicating compressions or other regular behavior, while a wider peak may indicate irregular motion of the chest and more random noise.

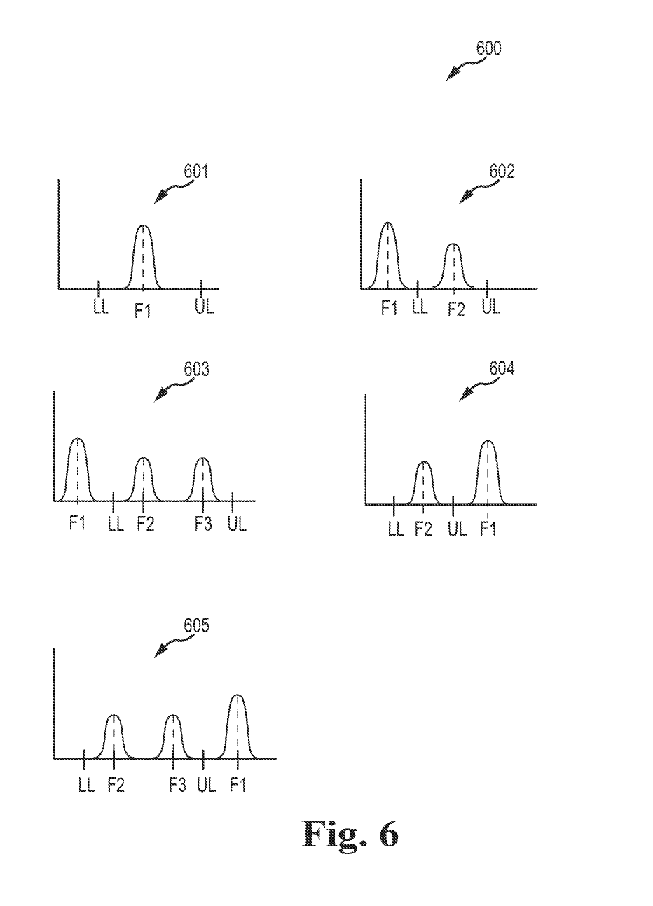

The CPR analyzer 242 may identify the highest peak from the detected peaks. The CPR analyzer 242 may classify the clip as CPR (e.g., classified as having CPR chest compressions based on the peak data) if the identified highest peak is within a defined frequency range. FIG. 6 depicts an example 601 having a highest peak at frequency Fl within the defined frequency range between the lower limit (LL) and the upper limit (UL). The frequency of the identified highest peak may be used by the CPR analyzer 242 as the chest compression rate. The defined frequency range may be a range that is consistent with chest compression frequency. For example, the defined frequency range may be between 1.2 Hz and 3 Hz. The defined frequency range may have a tolerance outside of the defined frequency range if a peak starts in the defined frequency range. For example, the tolerance may be half of the bandwidth threshold (e.g., 0.3 Hz when the bandwidth threshold is a 0.6 Hz).

If the identified highest peak is outside the defined frequency range (e.g., plus the tolerance), the CPR analyzer 242 may identify additional, secondary peaks that are located within the defined frequency range from the detected peaks and have an amplitude that meets a secondary peak amplitude threshold. In some examples, the secondary peak amplitude threshold may be 55% of the amplitude of the highest peak. The highest peak may fall outside the desired frequency range due to other environmental factors, such as ventilation performed during CPR or a rebound effect of the chest compressions.

If the identified highest peak is below the defined frequency range and an identified secondary peak is within the defined frequency range, the CPR analyzer 242 may classify the clip as CPR, and the CPR analyzer 242 may use the frequency of the identified secondary peak as the chest compression rate. In this example, the CPR analyzer 242 may set the ventilation rate as the frequency of the identified highest peak. FIG. 6 depicts an example 602 having a highest peak at frequency F1 below the defined frequency range LL-UL, and a secondary peak at frequency F2 within the defined frequency range LL-UL. If two secondary peaks are identified within the defined frequency range, the CPR analyzer 242 may determine whether one secondary peak is half of the frequency of the other secondary peak (e.g., within the tolerance). If so, the CPR analyzer 242 may classify the clip as CPR, and may use the frequency of the lower-frequency secondary peak as the chest compression rate. FIG. 6 depicts an example 603 having a highest peak at frequency F1 below the defined frequency range LL-UL, and a first secondary peak at frequency F2 and a second secondary peak at frequency F3 both within the defined frequency range LL-UL. In the example 603, the first secondary peak at frequency F2 may be selected for the chest compression rate. If the identified highest peak is below the defined frequency range and there are no secondary peaks within the defined frequency range, the CPR analyzer 242 may classify the clip as no CPR (e.g., having no CPR chest compressions).

If the identified highest peak is above the defined frequency range and an identified secondary peak has a frequency that is half of the frequency (e.g., within the tolerance) of the identified highest peak, the CPR analyzer 242 may classify the clip as CPR, and the CPR analyzer 242 may use the frequency of the identified secondary peak as the chest compression rate. FIG. 6 depicts an example 604 having a highest peak at frequency F1 above the defined frequency range LL-UL, and a secondary peak at frequency F2 within the defined frequency range LL-UL. If the secondary peak at frequency F2 is half of the frequency Fl, then the clip may be classified as CPR. Otherwise, it may be classified as no CPR. In some examples, if two secondary peaks are identified within the defined frequency range; the CPR analyzer 242 may determine whether the frequency of the highest peak is a multiple of each of the secondary peak frequencies. If so, the CPR analyzer 242 may classify the clip as CPR, and may use the frequency of the lower-frequency secondary peak as the chest compression rate. In other examples, the CPR analyzer 242 may determine whether the frequency of the highest peak is a multiple of only one of the secondary peak frequencies, and if so, may classify the clip as CPR and may use that secondary peak frequency as the rate. FIG. 6 depicts an example 605 having a highest peak at frequency F1 above the defined frequency range LL-UL, and a first secondary peak at frequency F2 and a second secondary peak at frequency F3 both within the defined frequency range LL-UL. In one example, if the first frequency F1 is a multiple of each of the first and second secondary peaks at frequencies F2 and F3, then the clip may be classified as CPR. Otherwise, it may be classified as no CPR. In another example, if the first frequency F1 is a multiple of one of the first or second secondary peaks at frequencies F2 and F3, then the clip may be classified as CPR, and the rate may be set to the frequency associated with the one of the first or second secondary peaks. Otherwise, it may be classified as no CPR. If the identified highest peak is above the defined frequency range and there are no secondary peaks within the defined frequency range, the CPR analyzer 242 may classify the clip as no CPR.

In some examples, the CPR analyzer 242 may consider additional parameters associated with each clip to determine whether the data is sufficient or reliable enough to render a CPR classification (e.g., whether the CPR classification based on the peaks should be overridden). For example, the CPR analyzer 242 may determine an energy (e.g., a sum of squares) of the bandpass filtered clip, and if the energy is below a threshold, the clip may be classified as no CPR. The CPR analyzer 242 may also apply a high pass filter to the detrended clip, and if a ratio of the bandpass filtered clip to the energy of the high pass filtered clip falls below a threshold, the clip may be classified as no CPR. Further, the CPR analyzer 242 classify the clip as no CPR if a ratio of the high pass filtered clip to the energy of the detrended signal clip falls below a threshold. The maximum peak-to-peak measurement may also be analyzed by the CPR analyzer 242, and if it exceeds a threshold (e.g., because the signal has large noise artifacts), the clip may be classified as no CPR.

The CPR analyzer 242 may further analyze previous data to determine whether to classify the clip passed on the CPR/no CPR classification or override the classification based on the peak data to classify as no CPR. For example, if the standard deviation of the current clip is far greater or less (e.g., 300% more or less) than a rolling standard deviation average of previous clips that were classified as CPR and were in close temporal proximity to the current clip, the CPR analyzer 242 may classify the clip as no CPR administered regardless of the CPR/no CPR classification based on the peak data. In practice, a sharp, but sustained change in standard deviation may occur if a different responder begins administering CPR. In that case, the rolling average will eventually incorporate the new standard deviation value and the CPR analyzer 242 may start classifying clips as based on the CPR/no CPR classification based on the peak data. Further, if the CPR analyzer 242 indicates peak CPR for a clip, but the immediately previous and immediately subsequent clips are each classified as no CPR, then the current clip is also classified as no CPR. This elimination removes a spurious CPR classification that is likely in error. The CPR analyzer 242 may store the CPR parameters 262, such as the CPR classifications, historical clip data, rolling standard deviation average, bandwidth threshold, tolerance, etc., in the memory 264.

The algorithm performed by the controller 240 and the CPR analyzer 242 is exemplary. The order of steps and/or the thresholds and tolerances are exemplary. Additional steps or different steps may be used to in the CPR classification based on analysis of the impedance signal by the CPR analyzer 242.

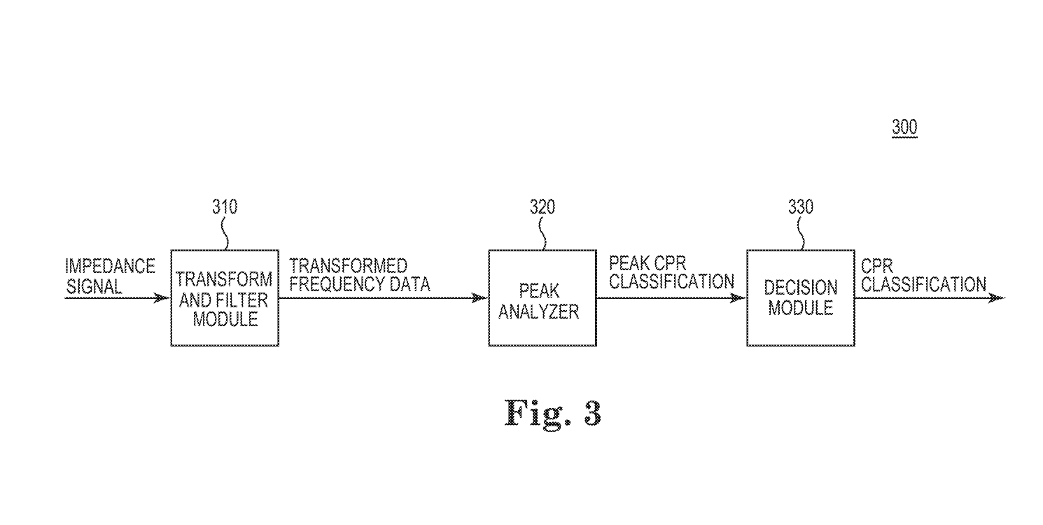

FIG. 3 is a block diagram of a CPR analyzer 300 according to an embodiment of the disclosure. The CPR analyzer 300 may be implemented in the CPR analyzer 242 of FIG. 2. The CPR analyzer 300 may include a filter and transform module 310, a peak analyzer 320, and a decision module 330. The filter and transform module 310 may receive an impedance signal clip and may filter the clip and transform the bandpass filtered clip to a frequency domain representation to provide transformed frequency data. The peak analyzer 320 may detect peaks within the transformed frequency data that meet a bandwidth threshold, and then analyze the detected peaks to determine whether to classify the transformed frequency data as CPR or no CPR. The peak analyzer 320 may provide the peak CPR classification to the decision module 330. The decision module 330 may perform additional statistical analysis of impedance signal data to determine whether to classify the impedance signal as CPR/no CPR based on the CPR classification from the peak analyzer 320, or to override the peak analyzer classification and to classify the impedance signal as no CPR.

In operation, the CPR analyzer 300 may analyze clips of the impedance signal, and may classify each clip as CPR or no CPR. The clips may overlap or may be independent. In an example, the CPR analyzer 300 may execute the CPR classification algorithm at one second intervals, which each clip being 6 seconds long. The CPR classification may be allocated to a mid-point of the respective clip (e.g., a middle one second of the clip when executed every second).

The filter and transform module 310 may filter and transform each clip. For example, the filter and transform module 310 may apply an amplitude weighting window function to the clip to reduce a side lobe effect. The filter and transform module 310 may detrend the windowed clip to remove a DC offset and linear trend. The filter and transform module 310 may apply a bandpass filter to the detrended clip to remove artifacts from the clip outside the pass frequencies. The filter and transform module 310 may transform the clip to the frequency domain by applying a discrete Fourier transform (DFT) to the bandpass filtered clip to provide transformed frequency data.

The peak analyzer 320 may detect all of the peaks that meet a bandwidth threshold within the transformed frequency data. The bandwidth threshold may be based on a bandwidth between points of the peak. For example, the bandwidth threshold may be a half-amplitude bandwidth, and the identified peak may have a half-amplitude bandwidth less than the bandwidth threshold. In an example, the bandwidth threshold may be 0.6 Hz.

The peak analyzer 320 may identify the highest peak from the detected peaks. The peak analyzer 320 may classify the clip as CPR if the identified highest peak is within a defined frequency range. The frequency of the identified highest peak may be used by the peak analyzer 320 as the chest compression rate. The defined frequency range may have a tolerance outside of the defined frequency range if a peak starts in the defined frequency range. For example, the tolerance may be half of the bandwidth threshold (e.g., 0.3 Hz when the bandwidth threshold is a 0.6 Hz).

If the identified highest peak is outside the defined frequency range (e.g., plus the tolerance), the peak analyzer 320 may identify additional, secondary peaks that are located within the defined frequency range from the detected peaks. Each of the identified secondary peaks may have an amplitude of at least 55% of the highest peak.

If the identified highest peak is below the defined frequency range and an identified secondary peak is within the defined frequency range, the peak analyzer 320 may classify the clip as CPR, and the peak analyzer 320 may use the frequency of the identified secondary peak as the chest compression rate. In this example, the CPR analyzer 320 may set the ventilation rate as the frequency of the identified highest peak. If two secondary peaks are identified within the defined frequency range, the peak analyzer 320 may determine whether one secondary peak is half of the frequency of the other secondary peak (e.g., within the tolerance). If so, the peak analyzer 320 may classify the clip as CPR, and may use the frequency of the lower-frequency secondary peak as the chest compression rate. If the identified highest peak is below the defined frequency range and there are no secondary peaks within the defined frequency range, the peak analyzer 320 may classify the clip as no CPR.

If the identified highest peak is above the defined frequency range and an identified secondary peak has a frequency that is half of the frequency (e.g., within the tolerance) of the identified highest peak, the peak analyzer 320 may classify the clip as CPR, and the peak analyzer 320 may use the frequency of the identified secondary peak as the chest compression rate. In some examples, if two secondary peaks are identified within the defined frequency range, the peak analyzer 320 may determine whether each of the secondary peak frequencies are multiples of the frequency of highest identified peak. If so, the peak analyzer 320 may classify the clip as CPR, and may use the frequency of the lower-frequency secondary peak as the chest compression rate. In other examples, the peak analyzer 330 may determine whether the frequency of the highest peak is a multiple of only one of the secondary peak frequencies, and if so, may classify the clip as CPR and may use that secondary peak frequency as the rate. If the identified highest peak is above the defined frequency range and there are no secondary peaks within the defined frequency range, the peak analyzer 320 may classify the clip as no CPR. The peak analyzer 320 may provide the CPR classification to the decision module 330.

In some examples, the decision module 330 may analyze consider additional parameters associated with each clip to determine whether the data is sufficient or reliable enough to render a CPR classification (e.g., whether the CPR classification based on the peaks should be overridden). For example, the decision module 330 may determine an energy (e.g., a sum of squares) of the bandpass filtered clip, and if the energy is below a threshold, the clip may be classified as no CPR. The decision module 330 may also apply a high pass filter to the detrended clip, and if a ratio of the bandpass filtered clip to the energy of the high pass filtered clip falls below a threshold, the clip may be classified as no CPR. Further, the decision module 330 classify the clip as no CPR if a ratio of the high pass filtered clip to the energy of the detrended signal clip falls below a threshold. The maximum peak-to-peak measurement may also be analyzed by the decision module 330, and if it exceeds a threshold (e.g., because the signal has large noise artifacts), the clip may be classified as no CPR.

The decision module 330 may further analyze previous data in comparison with the impedance signal clip to determine whether to classify the clip based on the CPR classification from the peak analyzer 320 or to override the CPR classification from the peak analyzer 320 and classify the clip as no CPR. For example, if the standard deviation of the current clip is far greater or less than a rolling standard deviation average of previous clips that were classified as CPR and were in close temporal proximity to the current clip, the decision module 330 may classify the clip as no CPR administered regardless of the CPR classification from the peak analyzer 320. Further, if the peak analyzer 320 indicates CPR for a current clip, but the immediately previous and immediately subsequent clips are each classified as no CPR, then the current clip is also classified as no CPR.

Generally, the CPR analyzer 300 may be implemented any device capable of receiving and processing impedance signal data from electrodes placed across the chest of a patient, including a real time analysis (e.g., during a medical event) or a post hoc analysis to assess CPR quality or determine CPR quality metrics for a single patient or as part of an analysis to identify trends associated with CPR quality. That is, the CPR analyzer 300 may be used in an automatic external defibrillator, other defibrillators (e.g., as a manual defibrillator, an implantable defibrillator, a paramedic defibrillator, and/or a clinical defibrillator), and/or in any computing system capable of receiving/retrieving impedance signal data.

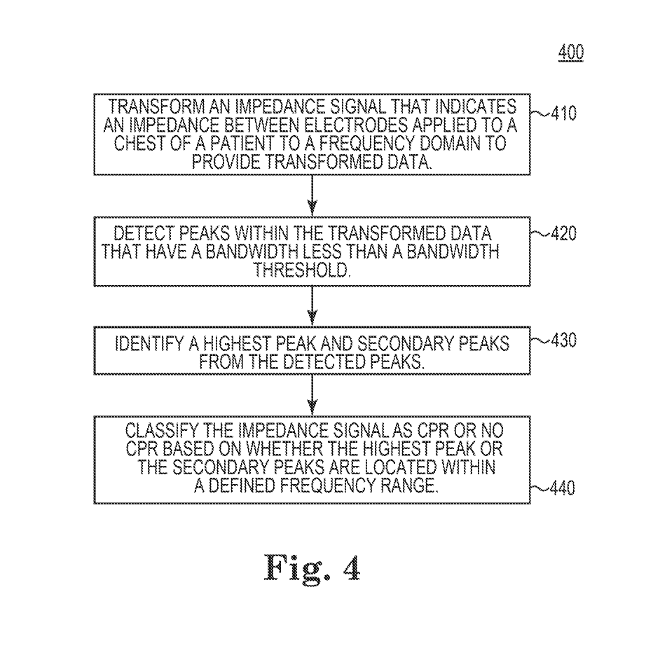

FIG. 4 is a flow chart of an exemplary method 400 according to the present disclosure. The method 400 may be implemented in the AED 100 of FIG. 1, the controller 240, the CPR analyzer 242, and/or the memory 260 of FIG. 2, the CPR analyzer 300 of FIG. 3, or any combination thereof.

The method 400 may include transforming an impedance signal that indicates an impedance between electrodes applied to a chest of a patient to a frequency domain representation to provide transformed frequency data, at 410. The transformation may be performed by the AED 110 of FIG. 1, the CPR analyzer 242 of FIG. 2, and/or the filter and transform module 310 of FIG. 3. In some embodiments, the method 400 may further include applying a window function to the impedance signal and applying a bandpass filter to the windowed impedance signal. In some examples, the transformation of the impedance signal may be based on the bandpass filtered signal. The transformation may include applying a DFT to the bandpass filtered signal.

The method 400 may further include detecting peaks within the transformed frequency data, wherein the peaks have a bandwidth that is less than a bandwidth threshold, at 420. The peak detection may be performed by the AED 110 of FIG. 1, the CPR analyzer 242 of FIG. 2, and/or the peak analyzer 310 of FIG. 3. In some embodiments, the detected peaks may meet a bandwidth threshold. In an example, the bandwidth threshold is a half-amplitude bandwidth threshold. The method 400 may further include identifying a highest peak and secondary peaks, at 430.

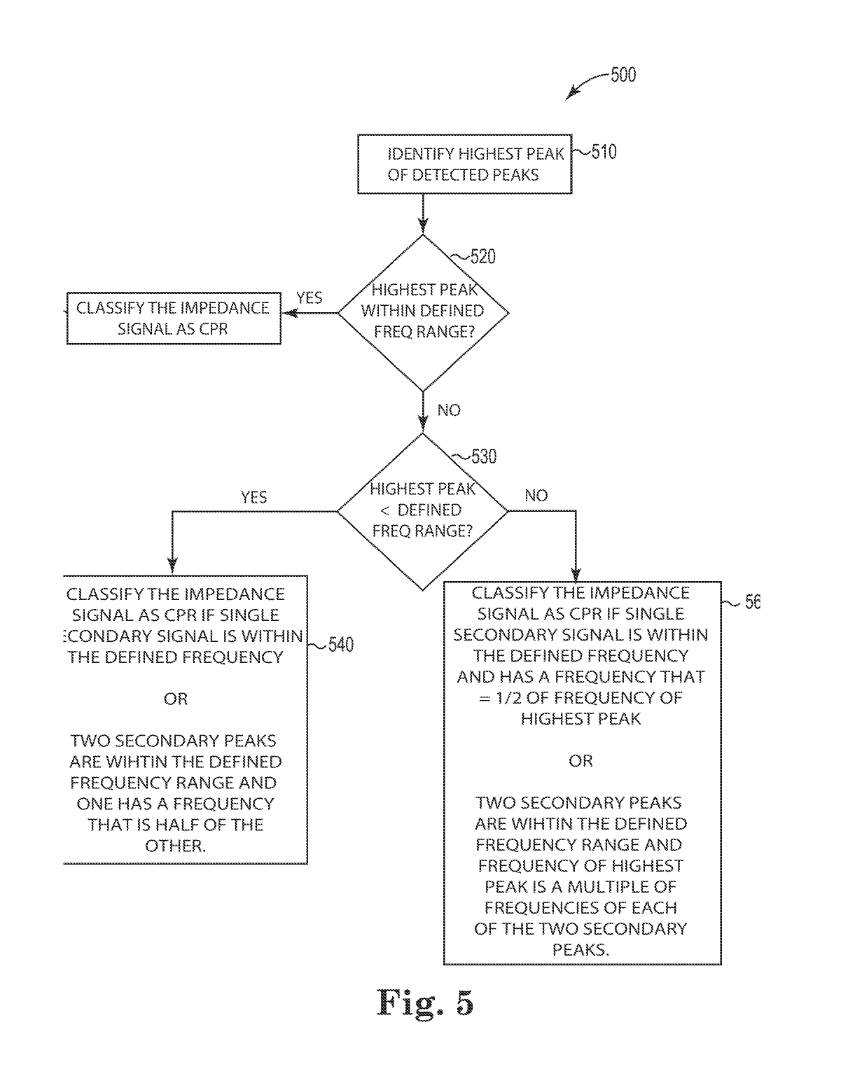

The method 400 may further include classifying the impedance signal as CPR or no CPR based whether the highest peak or the secondary peaks are located within a defined frequency range, at 440. The classification based on peak locations may be performed by the AED 110 of FIG. 1, the CPR analyzer 242 of FIG. 2, and/or the peak analyzer module 320 of FIG. 3. FIG. 5 provides an exemplary flowchart 500 for classifying the impedance signal based on peak locations. For example, the impedance signal may be classified as CPR, at 525, when a highest detected peak is within a defined frequency range, at 520, and the chest compression rate may be the frequency of the highest peak. When a highest detected peak is below a defined frequency range, at 530, and a secondary peak is located within the defined frequency range, the impedance signal may be classified as CPR, 540. If the highest peak is below the defined frequency range, at 530, and two secondary peaks are identified within the defined frequency range and one secondary peak is half of the frequency of the other secondary peak, the impedance signal may be classified as CPR, at 540, and the chest compression rate may be set to the frequency of the lower-frequency secondary peak. In this example, the method 400 may include setting the artificial ventilation rate to the frequency of the highest peak. Further, the impedance signal may be classified as CPR when a highest detected peak is above a defined frequency range, at 530, and a secondary peak is located within the defined frequency range at a frequency that is half of a frequency of the highest detected peak, at 560. If the highest peak is above the defined frequency range, at 530, and two secondary peaks are identified within the defined frequency range and the frequency of the highest peak is a multiple of each of the secondary peaks, the impedance signal may be classified as CPR, at 560, and the chest compression rate may be set to the frequency of the lower-frequency secondary peak. In another example, if the highest peak is above the defined frequency range, at 530, and two secondary peaks are identified within the defined frequency range and the frequency of the highest peak is a multiple of one of the secondary peaks, the impedance signal may be classified as CPR, and the chest compression rate may be set to the frequency of the identified one of the secondary peaks.

In some embodiments, the method 400 may further include classifying the impedance signal as no CPR when the classification of a previous impedance signal is no CPR and the classification of a subsequent impedance signal is no CPR. In some embodiments, the method 400 may further include classifying the impedance signal as no CPR when a standard deviation of the impedance signal exceeds or falls below a rolling average of the standard deviation of the impedance signals that were classified as CPR, and were in close temporal proximity to the current clip, by a defined amount. The classification based on previous statistical data may be performed by the AED 110 of FIG. 1, the CPR analyzer 242 of FIG. 2, and/or the decision module 330 of FIG. 3.

In some examples, the method 400 may further include overriding other classifications of the impedance signal, and classifying the impedance signal as no CPR based on analysis of the parameters associated with the impedance signal. For example, the method 400 may include determining an energy (e.g., a sum of squares) of the bandpass filtered signal, and if the energy is below a threshold, classifying the impedance signal as no CPR. In some examples, the method 400 may further include applying a high pass filter to the detrended signal, and if a ratio of the bandpass filtered signal to the energy of the high pass filtered signal falls below a threshold, classifying the impedance signal no CPR. In some examples, the method 400 may further include, classifying the impedance signal as no CPR if a ratio of the high pass filtered signal to the energy of the detrended signal falls below a threshold. In some examples, the method 400 may further include determining a maximum peak-to-peak measurement, and if the maximum peak-to-peak measurement exceeds a threshold (e.g., because the signal has large noise artifacts), classifying the clip as no CPR.

The method 400 and/or the flowchart 500 may be implemented by a field-programmable gate array (FPGA) device, an application-specific integrated circuit (ASIC), a processing unit such as a central processing unit (CPU), a digital signal processor (DSP), a controller, another hardware device, a firmware device, or any combination thereof. As an example, the method 400 and/or the flowchart 500 may be implemented by a computing system using, for example, one or more processing units that may execute instructions for performing the method that may be encoded on a computer readable medium. The processing units may be implemented using, e.g. processors or other circuitry capable of processing (e.g. one or more controllers or other circuitry). The computer readable medium may be transitory or non-transitory and may be implemented, for example, using any suitable electronic memory, including but not limited to, system memory, flash memory, solid state drives, hard disk drives, etc. One or more processing units and computer readable mediums encoding executable instructions may be used to implement all or portions of noise filter systems, encoders, and/or encoding systems described herein.

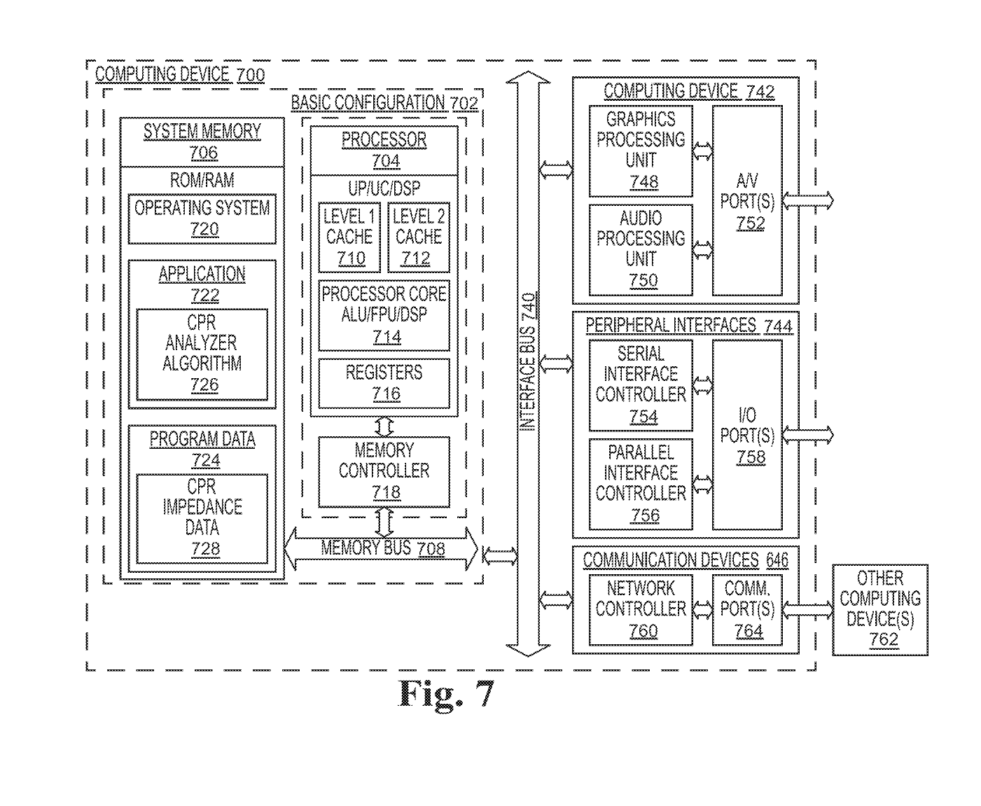

FIG. 7 is a block diagram illustrating an example computing device 700 that is arranged to implement remote display control according to at least some embodiments described herein. In a very basic configuration 702, computing device 700 typically includes one or more processors 704 and a system memory 706. A memory bus 708 may be used for communicating between processor 704 and system memory 706.

Depending on the desired configuration, processor 704 may be of any type including but not limited to a microprocessor (μP), a microcontroller (μC), a digital signal processor (DSP), or any combination thereof. Processor 704 may include one or more levels of caching, such as a level one cache 710 and a level two cache 712, a processor core 714, and registers 716. An example processor core 714 may include an arithmetic logic unit (ALU), a floating point unit (FPU), a digital signal processing core (DSP Core), or any combination thereof. An example memory controller 718 may also be used with processor 704, or in some implementations memory controller 718 may be an internal part of processor 704.

Depending on the desired configuration, system memory 706 may be of any type including but not limited to volatile memory (such as RAM), non-volatile memory (such as ROM, flash memory, etc.) or any combination thereof. System memory 706 may include an operating system 720, one or more applications 722, and program data 724. Application 722 may include a CPR analyzer 726 that is arranged to perform the functions as described herein including those described with respect to the method 400 of FIG. 4 and/or the flowchart 500 of FIG. 5. The CPR analyzer 726 may include the AED 110 of FIG. 1, the controller 240, the CPR analyzer 242, and/or the CPR parameters 264 of FIG. 2, the CPR analyzer 300 of FIG. 3, or combinations thereof. Program data 724 may include CPR data 728 that may be useful for operation with the remote display control algorithm as is described herein. The CPR data 728 may include the CPR parameters 264 of FIG. 2, and/or data from the CPR analyzer 242 of FIG. 2 and/or the CPR analyzer 300 of FIG. 3. In some embodiments, application 722 may be arranged to operate with program data 724 on operating system 720 such that implementations of convenient remote display control may be provided as described herein. This described basic configuration 702 is illustrated in FIG. 7 by those components within the inner dashed line.

Computing device 700 may have additional features or functionality, and additional interfaces to facilitate communications between basic configuration 702 and any required devices and interfaces. For example, a bus/interface controller may be used to facilitate communications between basic configuration 702 and one or more data storage devices via a storage interface bus. Data storage devices may be removable storage devices, non-removable storage devices, or a combination thereof. Examples of removable storage and non-removable storage devices include magnetic disk devices such as flexible disk drives and hard-disk drives (HDD), optical disk drives such as compact disk (CD) drives or digital versatile disk (DVD) drives, solid state drives (SSD), and tape drives to name a few. Example computer storage media may include volatile and nonvolatile, removable and non-removable media implemented in any method or technology for storage of information, such as computer readable instructions, data structures, program modules, or other data.

System memory 706, removable storage devices and non-removable storage devices are examples of computer storage media. Computer storage media includes, but is not limited to, RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which may be used to store the desired information and which may be accessed by computing device 700. Any such computer storage media may be part of computing device 700.

Computing device 700 may also include an interface bus 740 for facilitating communication from various interface devices (e.g., output devices 742, peripheral interfaces 744, and communication devices 746) to basic configuration 702 via bus/interface controller. Example output devices 742 include a graphics processing unit 748 and an audio processing unit 750, which may be configured to communicate to various external devices such as a display or speakers via one or more A/V ports 752. Example peripheral interfaces 744 include a serial interface controller 754 or a parallel interface controller 756, which may be configured to communicate with external devices such as input devices (e.g., keyboard, mouse, pen, voice input device, touch input device, etc.) or other peripheral devices (e.g., printer, scanner, etc.) via one or more I/O ports 758. An example communication device 746 includes a network controller 760, which may be arranged to facilitate communications with one or more other computing devices 762 over a network communication link via one or more communication ports 764.

The network communication link may be one example of a communication media. Communication media may typically be embodied by computer readable instructions, data structures, program modules, or other data in a modulated data signal, such as a carrier wave or other transport mechanism, and may include any information delivery media. A “modulated data signal” may be a signal that has one or more of its characteristics set or changed in such a manner as to encode information in the signal. By way of example, and not limitation, communication media may include wired media such as a wired network or direct-wired connection, and wireless media such as acoustic, radio frequency (RF), microwave, infrared (IR) and other wireless media. The term computer readable media as used herein may include both storage media and communication media.

Computing device 700 may be implemented as a portion of a small-form factor portable (or mobile) electronic device such as a cell phone, a personal data assistant (PDA), a personal media player device, a wireless web-watch device, a personal headset device, an application specific device, or a hybrid device that include any of the above functions. Computing device 700 may also be implemented as a personal computer including both laptop computer and non-laptop computer configurations.

It is intended that all matter contained in the above description or shown in the accompanying drawings shall be interpreted as illustrative only and not limiting. Changes in detail or structure may be made without departing from the spirit of the invention as defined in the appended claims. In addition, although various representative embodiments of this invention have been described above with a certain degree of particularity, those skilled in the art could make numerous alterations to the disclosed embodiments without departing from the spirit or scope of the inventive subject matter set forth in the specification and claims.

What is claimed is:

1. A system comprising:
a defibrillator comprising a cardiopulmonary resuscitation (CPR) analyzer configured to detect an impedance signal between electrodes applied to a chest of a patient, the CPR analyzer further configured to transform the impedance signal to a frequency domain representation to provide transformed frequency data, the CPR analyzer further configured to detect peaks within the transformed frequency data and to classify the impedance signal as one of CPR or no CPR based on the detected peaks.

2. The system of claim 1, wherein the defibrillator is further configured to apply a window function to the impedance signal prior to transforming the impedance signal to the frequency domain representation.

3. The system of claim 1, wherein the defibrillator is further configured to apply a bandpass filter to the impedance signal prior to transforming the impedance signal to the frequency domain representation.

4. The system of claim 1, wherein the defibrillator is configured to transform the impedance signal by applying a discrete Fourier transform to the impedance signal.

5. The system of claim 1, wherein the defibrillator configured to classify the impedance signal as one of CPR or no CPR based on the detected peaks comprises classification of the impedance signal as CPR when a highest detected peak is within a defined frequency range.

6. The system of claim 1, wherein the defibrillator configured to classify the impedance signal as one of CPR or no CPR based on the detected peaks comprises classification of the impedance signal as CPR when a highest detected peak is below a defined frequency range and a secondary peak is located within the defined frequency range.

7. The system of claim 1, wherein the defibrillator configured to classify the impedance signal as one of CPR or no CPR based on the detected peaks comprises classification of the impedance signal as CPR when a highest detected peak is below a defined frequency range and two secondary peaks are located within the defined frequency range and one of the two secondary peaks is half of the frequency of the other of the two secondary peaks.

8. The system of claim 1, wherein the defibrillator configured to classify the impedance signal as one of CPR or no CPR based on the detected peaks comprises classification of the impedance signal as CPR when a highest detected peak is above a defined frequency range and a secondary peak is located within the defined frequency range at a frequency that is half of a frequency of the highest detected peak.

9. The system of claim 1, wherein the defibrillator configured to classify the impedance signal as one of CPR or no CPR based on the detected peaks comprises classification of the impedance signal as CPR when a highest detected peak is above a defined frequency range and two secondary peaks are located within the defined frequency range and a frequency of the highest detected peak is a multiple of at least one of the two secondary peaks.

10. The system of claim 1, wherein the defibrillator configured to detect peaks within the transformed frequency data comprises detection of peaks have a bandwidth that meet a bandwidth threshold.

11. The system of claim 10, wherein the bandwidth is a half-amplitude bandwidth.

12. The system of claim 1, wherein the defibrillator is further configured to select an algorithm for analysis of an ECG signal based on the CPR or no CPR classification of the impedance signal.

13. A non-transitory computer-readable medium comprising instructions that, when executed by one or more processing units, cause the one or more processing units to:
transform an impedance signal that indicates impedance between electrodes applied to a chest of a patient to a frequency domain representation to provide transformed frequency data;
detect peaks within the transformed frequency data, wherein the peaks have a bandwidth that is less than a bandwidth threshold;
identify a highest peak and secondary peaks; and
classify the impedance signal as CPR or no CPR based whether the highest peak or the secondary peaks are located within a defined frequency range.

14. The non-transitory computer-readable medium of claim 13, further comprising instructions that, when executed by the one or more processing units, cause the one or more processing units to, prior to transformation of the impedance signal to the frequency domain representation:
apply a window function to the impedance signal;
detrend the windowed impedance signal; and
apply a bandpass filter to the detrended impedance signal.

15. The non-transitory computer-readable medium of claim 14, further comprising instructions that, when executed by the one or more processing units, cause the one or more processing units to:
classify the impedance signal as no CPR when an energy of the bandpass filtered signal is below a first threshold, wherein an energy of the bandpass filtered signal is a sum of squares of the bandpass filtered signal.

16. The non-transitory computer-readable medium of claim 14, further comprising instructions that, when executed by the one or more processing units, cause the one or more processing units to:
apply a high pass filter to the detrended impedance signal; and
classify the impedance signal as no CPR when a ratio of the energy of the bandpass filtered signal to an energy of the high pass filtered signal is below a second threshold, wherein an energy of the high pass filtered signal is a sum of squares of the high pass filtered signal.

17. The non-transitory computer-readable medium of claim 16, further comprising instructions that, when executed by the one or more processing units, cause the one or more processing units to classify the impedance signal as no CPR when a ratio of the energy of the high pass filtered signal to an energy of the detrended signal is below a third threshold, wherein an energy of the detrended signal is a sum of squares of the detrended signal.

18. The non-transitory computer-readable medium of claim 13, further comprising instructions that, when executed by the one or more processing units, cause the one or more processing units to classify the impedance signal as no CPR when a maximum peak-to-peak measurement of the transformed data exceeds a max peak-to-peak threshold.

19. The non-transitory computer-readable medium of claim 13, further comprising instructions that, when executed by the one or more processing units, cause the one or more processing units to classify the impedance signal as no CPR when a classification of a temporally immediately previous impedance signal is no CPR and a classification of an immediately subsequent impedance signal is no CPR.

20. The non-transitory computer-readable medium of claim 13, further comprising instructions that, when executed by the one or more processing units, cause the one or more processing units to classify the impedance signal as no CPR when a standard deviation of the impedance signal exceeds or falls below a rolling average of the standard deviations of previous impedance signals that were classified as CPR and were in close temporal proximity to the impedance signal by a defined amount.

21. The non-transitory computer-readable medium of claim 13, further comprising instructions that, when executed by the one or more processing units, cause the one or more processing units to:
classify the impedance signal as CPR when a highest detected peak is within a defined frequency range;
classify the impedance signal as CPR when a highest detected peak is below a defined frequency range and a secondary peak is located within the defined frequency range; and
classify the impedance signal as CPR when a highest detected peak is above a defined frequency range and a secondary peak is located within the defined frequency range at a frequency that is half of a frequency of the highest detected peak.

22. A method, comprising:
transforming a clip of an impedance signal that indicates impedance between electrodes applied to a chest of a patient to a frequency domain representation to provide transformed frequency data;
identifying, within the transformed frequency data, a highest peak having a bandwidth that meets a bandwidth threshold; and
responsive to the highest peak being located within a defined frequency range, classifying the clip as cardiopulmonary resuscitation (CPR); and
responsive to the highest peak being located outside the defined frequency range, classifying the clip as CPR or no CPR based on secondary peak located within the defined frequency range.

23. The method of claim 22, further comprising identifying, within the transformed frequency data, the secondary peaks, wherein the secondary peaks have an amplitude of at least a secondary peak amplitude threshold associated with an amplitude of the highest peak.

24. The method of claim 23, wherein the secondary peak amplitude threshold is 55% of the amplitude of the highest peak.

25. The method of claim 22, wherein the secondary peaks have a bandwidth that meets the bandwidth threshold.

26. The method of claim 22, further comprising, responsive to the highest peak and all secondary peaks being located outside the defined frequency range, classifying the clip no CPR.

27. The method of claim 22, further comprising, responsive to a CPR classification, determining a chest compression rate that is based on:
a frequency of the highest peak when the highest peak is located within the defined frequency range; and
a frequency of one of the secondary peaks located within the defined frequency range when the highest peak is located outside of the defined frequency range.

28. The method of claim 22, further comprising, responsive classification of the clip as no CPR, providing an alert to resume CPR.
